(19) United States
(12) Patent Application Publication
(10) Pub. No.: US 2004/0023133 A1
(43) Pub. Date: Feb. 5, 2004

(54) Photoresist pattern and forming method thereof

(75) Inventors: Hiroshi Shimbori, Kawasaki-shi (JP); Takako Hirosaki, Kawasaki-shi (JP)

Correspondence Address: ALLEN BLOOM, C/O DECHERT, PRINCETON PIKE CORPORATION CENTER, P.O. BOX 5218, PRINCETON, NJ, 08543-5218, US

(73) Assignee: Tokyo Ohka Kogyo Co., Ltd.

(21) Appl. No.: 10/628,919

(22) Filed: Jul. 29, 2003

(30) Foreign Application Priority Data
Jul. 31, 2002 (JP) 2002-223984

(51) Int. Cl. G03F 1/16; G03F 7/20; G03F 7/30; B32B 3/10; G03F 9/00; G21G 5/00; A61N 5/00

(52) U.S. Cl. 430/5; 430/17; 430/296; 250/492.3; 430/311; 428/195.1

(57) ABSTRACT

A photoresist pattern with a reinforcing section, provided with a line section 2 and a reinforcing section 3 that continues to the line section and that has a greater width than a line width of the line section is formed by forming on a substrate a photoresist film, exposing the photoresist film and then developing. As a result, fine line shaped photoresist patterns are prevented from collapsing and being washed away.

### BACKGROUND OF THE INVENTION

1. Field of the Invention

The present invention relates to a method of forming a photoresist pattern.

2. Description of the Related Art

In the manufacture of fine structures in a range of devices such as semiconductor devices, and the like, lithographic methods are used, and similar fineness is required in the lithographic processes to match the fineness of the device structures.

Currently, there is a case where fine patterns with line widths of 0.20 μm or less are formed by lithographic methods. In this case, if a fine line shaped photoresist pattern is formed in this manner, the self-supportability of the photoresist pattern deteriorates, so that there are problems in that it may be washed away into a developing solution in the developing process stage, or it may collapse, or the like, even if it remains after the developing process.

In a device manufacturing process, if the line shaped photoresist pattern is washed away or collapses in this manner, a photoresist pattern of the intended shape cannot be obtained, which causes a failure of manufacturing. Hence there is a problem of a drop in yield.

Furthermore, a typical method used for evaluating photoresist patterns is one in which a line shaped photoresist pattern is formed on a substrate, which extends in the vertical direction to the cleavage plane of the substrate to create an evaluation substrate, and the evaluation substrate is cut at the cleavage plane to observe the cross section of the photoresist pattern. However, when this method is used, if the photoresist pattern is washed away or collapsed, there is also a problem that observation and evaluation of the cross section cannot be performed correctly.

Moreover, in particular, in a case where a sublayer contacting the photoresist pattern is formed from a layer soluble in the developing solution for the photoresist pattern, such as in an implantation process, a lift-off pattern forming process or the like, it is easy to damage the integrity of the photoresist pattern in the developing process.

### SUMMARY OF THE INVENTION

Accordingly, an object of the present invention is to prevent a fine line shaped photoresist pattern from collapsing and being washed away.

In order to solve the above-described problems, the present invention provides a photoresist pattern with a reinforcing section, wherein there is provided a line section and a reinforcing section that continues to the line section and that has a greater width than a line width of the line section.

Furthermore, the present invention provides a method of forming a photoresist pattern, comprising forming the above-described photoresist pattern with a reinforcing section by a process comprising forming on a substrate a photoresist film, exposing the photoresist film, and then developing the photoresist film.

A sublayer film that is soluble in a developing solution used in the developing process may be formed on the substrate, and the photoresist film may be formed on the sublayer film.

Moreover, the present invention provides a method of evaluating a photoresist pattern, comprising forming on a substrate the above-described photoresist pattern with a reinforcing section, using the above-described photoresist pattern forming method to create an evaluation substrate, and splitting the evaluation substrate in a cross section perpendicular to the lengthwise direction of the line section to observe its cross section.

When creating the evaluation substrate, it is preferable that a plurality of photoresist patterns with reinforcing sections is formed such that lengthwise directions of line sections are parallel, and locations of reinforcing sections in the lengthwise direction of the line sections are different for adjacent photoresist patterns with reinforcing sections.

Furthermore, the present invention provides a method of manufacturing a device using a lithographic method that includes a process of forming on a substrate a photoresist pattern having a line section on at least part thereof, wherein the photoresist pattern is formed such that there is provided on the line section a reinforcing section having a greater width than the line width of the line section, or a plurality of such reinforcing sections with spaces between.

Moreover, the present invention provides a mask used when exposing a photoresist film formed on a substrate, wherein a shape of either one of an exposure region and a non exposure region of the mask comprises a succession of line sections, and reinforcing sections having a greater width than a line width of the line sections.

Furthermore, the present invention provides a writing system used when drawing on a photoresist film formed on a substrate, which comprises an electron beam direct writing system that is provided with: a holding device for holding the substrate; an irradiating device for irradiating an electron beam onto the substrate; a moving device for moving a location on the substrate irradiated by the electron beam; and a control device for creating pattern data to determine an irradiation region of the electron beam on the substrate according to input information, and for controlling the moving device and the irradiating device based on the pattern data, wherein the shape of the irradiation region of the electron beam is set to the shape of a succession of line sections, and reinforcing sections having a greater width than the line width of the line sections, and it is possible to input as the information, the line width of the line section, the length of the line section, the shape of the reinforcing section, the maximum width of the reinforcing section, and the length of the reinforcing section.

As described above, by using a photoresist pattern with a reinforcing section and a method of forming such a photoresist pattern of the present invention, it is possible to prevent a fine line shaped photoresist pattern from collapsing or being washed away in a photoresist pattern forming process.

A photoresist pattern with a reinforcing section and a method of forming the photoresist pattern of the present invention are ideal for use in a method of evaluating a photoresist pattern in which a photoresist pattern is formed on a substrate perpendicularly to the cleavage plane of the substrate to create an evaluation substrate, and the evaluation substrate is sectioned to observe the cross section of the photoresist pattern, and it is possible to prevent the photoresist pattern from collapsing or being washed away in a photoresist pattern forming process. Accordingly, it is also possible to observe and evaluate the shape of the cross section of a fine line shaped photoresist pattern accurately.

Furthermore, a photoresist pattern with a reinforcing section and a method of forming the photoresist pattern of the present invention are ideal for use in a method of manufacturing a device using a lithographic method including a process of forming a photoresist pattern, at least part of which is a line section, on a substrate, and it is also possible to prevent the photoresist pattern from collapsing or being washed away in a photoresist pattern forming process. As a result, it is possible to avoid the occurrence of failures in manufacturing and improve the yield.

### BRIEF DESCRIPTION OF THE DRAWINGS

FIG. 1 is a plan view showing an example of an evaluation substrate according to the present invention.

FIG. 2 is a plan view showing the main part of FIG. 1 enlarged.

FIG. 3 is a plan view showing an example of a mask according to the present invention.

### DETAILED DESCRIPTION OF THE PREFERRED EMBODIMENTS

Hereunder is a detailed description of preferred embodiments of the present invention.

In a first embodiment of the present invention, an example will be described in which a photoresist pattern with reinforcing sections, and a method of forming such a photoresist pattern, according to the present invention, are used to create an evaluation substrate, and the photoresist pattern is evaluated using this.

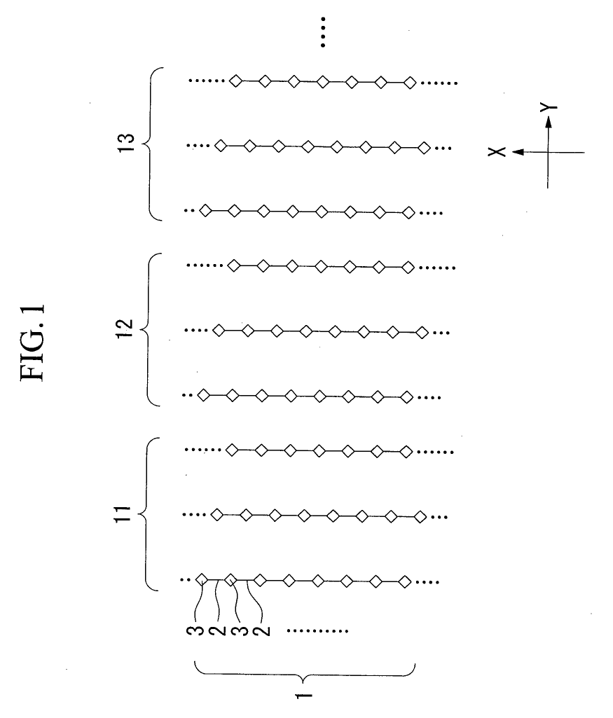

FIG. 1 is a plan view showing an example of a photoresist pattern formed on an evaluation substrate.

In the present embodiment, a photoresist pattern 1 with reinforcing sections is shaped as a series of alternate line sections 2 and square reinforcing sections 3, and adjacent reinforcing sections 3 are linked by a line section 2. A plurality of thus shaped photoresist patterns 1 with reinforcing sections is arranged such that the lengthwise direction of the line sections 2 are parallel. In the present embodiment, taking three photoresist patterns 1 with reinforcing sections, which are adjacent to each other, as one group, a plurality of groups of patterns 11, 12, 13 is formed on a substrate. The groups of patterns 11, 12, 13 differ from each other by at least one forming condition, and all three photoresist patterns 1 with reinforcing sections constituting each of the groups of patterns 11, 12, 13 are formed with the same forming conditions.

In the following description, the lengthwise direction of the line section 2 may be designated as the X direction, and the line widthwise direction of the line section 2 perpendicular to this as the Y direction.

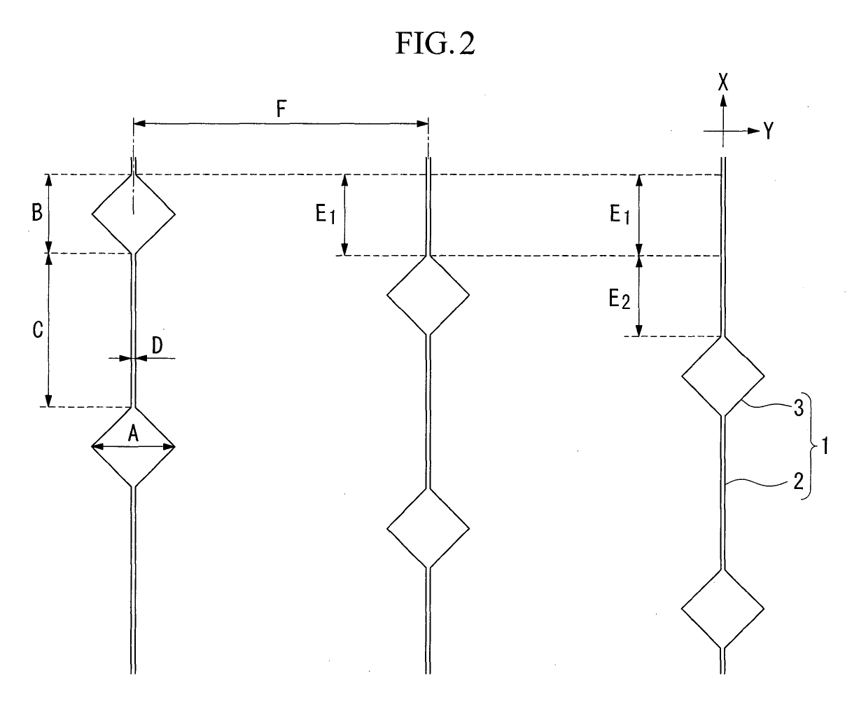

FIG. 2 shows the main part of a group of patterns in FIG. 1 enlarged. In the figure, numeral 1 denotes photoresist patterns with reinforcing sections.

The line width D of the line section 2 in the Y direction is set appropriately according to the application of the photoresist to be evaluated using this evaluation substrate, and it has no particular limitation.

The maximum width A of the reinforcing section 3, that is the maximum width of the reinforcing section 3 in the Y direction, may be greater than the line width D of the line section 2, and may be of sufficient size to support the line section 2 such that the line section 2 does not collapse and get washed away. It has no particular limitation. The greater the width A of the reinforcing section 3, the harder it is for the line section 2 to collapse. However, if it is too great, there is a concern that adjacent reinforcing sections 3 may overlap if photoresist patterns 1 with reinforcing sections are formed close together, hence it is preferable that the width A of the reinforcing section 3 is set to approximately 1.5 to 1000 times the line width D of the line section 2.

The reinforcing section 3 is square in the present embodiment. However, it is not limited to this, and can be any desired shape, such as a rhombus, rectangle, circle, ellipse or the like. Furthermore, it is also possible to set appropriately the connection of the line section on the periphery of the reinforcing section 3, but preferably the reinforcing section 3 is linearly symmetrical about the axis in the X direction which passes through the center of the line section 2 in plan view. The length B of the reinforcing section 3 in the X direction is equal to the width A of the reinforcing section 3 in the present embodiment. However it may be changed appropriately as long as the integrity of the reinforcing section 3 can be maintained stably even after passing through the developing process.

The longer the spacing between adjacent reinforcing sections 3 in a photoresist pattern 1 with reinforcing sections, that is the length C of the line section 2 in the X direction, the easier it is to cause the line section 2 between the reinforcing sections 3 to collapse or get washed away. Hence the length is set so as to maintain the self-supportability integrity of the reinforcing section 3 stably even after passing through the developing process. Furthermore, the length C of the line section 2 is adjusted appropriately taking the line width D of the line section and the thickness of the photoresist film into consideration.

A plurality of photoresist patterns 1 with reinforcing sections constituting a group of patterns 11 (12, 13) is arranged such that the reinforcing sections 3 are not adjacent to each other, that is the locations of the reinforcing sections 3 vary in the X direction in adjacent photoresist patterns 1 with reinforcing sections.

It is preferable to set the locations of the reinforcing sections 3 in a plurality of photoresist patterns 1 with reinforcing sections such that when the evaluation substrate is split in a cross section perpendicular to the X direction, one or more cross sections of the line sections 2 are contained in the cross section regardless of the location of the cross section in the X direction. Furthermore, it is desirable that the cross sections of the line sections 2 contained in the cross section are cross sections near the central part between the reinforcing sections 3 rather than cross sections of portions close to the reinforcing sections, in the line sections 2.

In the set of patterns shown as an example in FIG. 2, the positional difference E1 in the X direction between the location of the reinforcing section 3 of the far left photoresist pattern with reinforcing sections and the location of the reinforcing section 3 of the central photoresist pattern with reinforcing sections, and the positional difference E2 in the X direction between the location of the reinforcing section 3 of the central photoresist pattern with reinforcing sections and the location of the reinforcing section 3 of the far right photoresist pattern with reinforcing sections, are set to be equal.

It is preferable that the positional difference E1 (E2) is set within a range of approximately 10 to 50% of the total (B+C) of the length B of the reinforcing section 3 and the length C of the line section 2 in the X direction. Furthermore, where the number of photoresist patterns with reinforcing sections constituting a set of patterns is n, it is preferable that the positional difference E1 (E2) is set to be approximately (B+C)×(1/n).

Moreover, a spacing F between a plurality of photoresist patterns 1 with reinforcing sections constituting a set of patterns 11 (12, 13) may be the distance at which the reinforcing sections 3 do not make contact, or greater, and it may be set appropriately.

In order to create such an evaluation substrate, firstly, a photoresist to be evaluated using this evaluation substrate is coated onto the substrate by a known method, and is then pre-baked to form a photoresist film. The substrate may be of any type that is suitable for the application of photoresist, without particular limitation. For example, a silicon substrate, a glass substrate, an AlTiC substrate or the like can be used. For the photoresist, a positive type resist, or a negative type resist may be used.

Then, after the photoresist film formed on the substrate is exposed, heat treatment is performed, and a developing process is performed using a well known method to form the photoresist patterns 1 with reinforcing sections, thus an evaluating substrate may be obtained.

The photoresist film may be exposed using a mask, and it may also be formed without a mask using a writing system that draws directly by irradiating the photoresist film with an electron beam.

There is no particular limitation to the developing method, and it may be performed by dripping or spraying developing solution onto the substrate after exposure, and holding for a prescribed period.

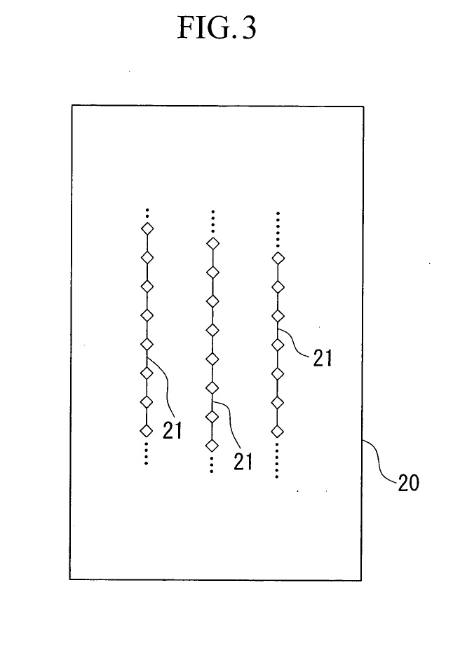

In the case of exposure using a mask, as shown in FIG. 3, a mask 20 is used that has a mask pattern 21 of the same shape as, or a similar enlarged shape to, the shape of the photoresist patterns 1 with reinforcing sections to be formed on the substrate. In the case where the photoresist is a negative type, the shape of the region on the mask through which light is irradiated, that is the shape of the exposed region in the mask, becomes the shape of the mask pattern. On the other hand, in the case where the photoresist is a positive type, the shape of the region on the mask through which light is irradiated, that is the shape of the non exposed region (enclosed region) in the mask, becomes the shape of the mask pattern.

Alternatively, it is possible to construct a writing system to be used for drawing directly by an electron beam, using an electron beam direct writing system having a holding device for holding a substrate on which a photoresist film is formed, an irradiation device for irradiating an electron beam onto the substrate, a moving device for moving the location where the electron beam is irradiated on the substrate, and a control device for creating pattern data to determine the location on the substrate irradiated by the electron beam according to the input information, and for controlling the moving device and the irradiating device based on the pattern data. The moving device comprises a device for moving the substrate, a device for moving the irradiating device, or both. The control device is constructed such that it controls the ON/OFF switching of the irradiating device, the amount of irradiation, and it also controls the moving device.

Furthermore, this writing system is programmed in advance such that the shape of the irradiation region of the electron beam is the shape of photoresist patterns 1 with reinforcing sections to be formed on a substrate, that is, pattern data is created, which corresponds to the shape of a succession of a line shaped line section and a reinforcing section having a greater width than the line width of the line section. The control device is constructed such that a line width D, a length C of the line section, the shape of the reinforcing section, a maximum width A of the reinforcing section and a length B of the reinforcing section can be set by external inputs. Furthermore, it is constructed such that in the case of creating a plurality of photoresist patterns 1 with reinforcing sections, the positional difference E1 (E2) between the reinforcing sections 3, the spacing between the photoresist patterns 1 with reinforcing sections, the number of the photoresist patterns 1 with reinforcing sections constituting a group of patterns, and the number of groups of patterns formed on the substrate can be set by external inputs. Moreover, it is preferable that the irradiation amount for each photoresist pattern 1 with reinforcing sections can be set by external inputs.

Such a writing system can be constructed using an existing electron beam direct writing system, and by providing a control device programmed such that pattern data corresponding to the photoresist patterns 1 with reinforcing sections are created based on the input information.

Furthermore, before forming a photoresist film on the substrate, another layer may be formed as a sublayer if required. For example, in the case where the photoresist to be evaluated using this evaluation substrate is used for an implantation process or a lift-off pattern forming process, a sublayer provided under the photoresist film in an actual process is soluble in the developing solution of the photoresist pattern. Therefore, in such a case it is preferable that even when creating an evaluation substrate, a sublayer that is soluble in the developing solution is formed on the substrate, and the photoresist film is formed on this to form the photoresist patterns 1 with reinforcing sections.

For such a sublayer soluble in the developing solution of a photoresist pattern, a commercial sublayer material can be used.

Such a sublayer can be formed by coating with a coating liquid containing film forming constituents on a substrate, and pre-baking it using a well known method.

In order to evaluate the project using an evaluation substrate created in this manner, the evaluation substrate obtained is split at a plane perpendicular to the lengthwise direction of the line section 2 (plane perpendicular to the X direction), and the cross section is observed using an appropriate observation device such as a scanning electron microscope (SEM).

There is no particular limitation to the method for splitting an evaluation substrate. However, in the case where the evaluation substrate is created using a silicon substrate, since the silicon substrate is easily split at a cleavage plane, the evaluation substrate can be easily split by hand.

According to the present embodiment, by observing a cross section of an evaluation substrate, it is possible to observe the cross section of a line section 2 of a photoresist pattern formed. Furthermore, in the case where a sublayer is provided, it is also possible to observe the condition of the cross section of the sublayer after the developing process.

In the case where a plurality of photoresist patterns 1 with reinforcing sections is provided on the evaluation substrate, it is necessary to form photoresist patterns 1 constituting at least one group of patterns 11 (12, 13) under the same conditions. However, if conditions such as the line width of a line section 2, the exposure (amount of electron beam irradiation) and the like are changed for each group of patterns 11, 12, 13, it is possible to evaluate the differences in the shapes of cross sections due to the differences in conditions. Furthermore, when creating a plurality of evaluation substrates, if the film thickness, baking condition, or developing condition is changed for each evaluation substrate, it is possible to evaluate the differences in the shapes of the cross sections due to the differences in the conditions.

According to the present embodiment, when exposing and developing a photoresist film to form a photoresist pattern, since the shape is such that both ends of a line section 2 are attached to a wide reinforcing section 3, then even if the line width of the line section 2 is small, the line section 2 neither collapses nor is washed away in the developing process. Hence it is possible to evaluate the shape of the cross section of the fine line pattern. Furthermore, especially in the case where a sublayer soluble in the developing solution is provided under the photoresist pattern, the integrity of the photoresist pattern may easily be unstable. However, according to the present embodiment, even if side etching occurs on the sublayer due to the developing process, or even if the sublayer is removed completely, since both ends of the line section 2 are attached to the reinforcing section 3, the line section is prevented from being washed away.

Furthermore, in the present embodiment, since it is formed such that the reinforcing sections 3 of the three photoresist patterns 1 constituting each group of patterns 11, 12 and 13 are not adjacent to each other, one cross section of at least one line section 2 on the cross section of each group of patterns 11 (12, 13) is included regardless of the location in the X direction of the cross section of an evaluation substrate split by hand. Hence it is possible to evaluate each of the patterns 11, 12 and 13, without exception.

In addition, in the present embodiment, there is provided a plurality of groups of patterns 11, 12, 13 whose forming conditions are different, on a substrate. However, the forming conditions of the plurality of photoresist pattern 1 formed on the substrate may be all the same.

Furthermore, in the present embodiment, the number of photoresist pattern 1 with reinforcing sections constituting a group of patterns is three, however it may be one, or may be any desired plurality. However, if it is one, there is a possibility that there is no cross section of a line section 2 included in the cross section, when an evaluation substrate is split.

Moreover, in the case where a plurality of photoresist pattern 1 with reinforcing sections is arranged so as to be parallel on an evaluation substrate, a construction may be possible wherein the locations of the reinforcing sections 3 are not shifted in the X direction, that is a construction wherein the positional difference E1 (E2) between the reinforcing sections 3 is zero. However, in this case, there is also a possibility that there is no cross section of a line section 2 included in the cross section when an evaluation substrate is split.

As a second embodiment of the present invention, an example of a manufacturing method of a device according to the present invention will be described.

A photoresist pattern with reinforcing sections and a method of forming such a photoresist pattern according to the present invention can be applied to a process, in a device manufacturing process, of forming a photoresist pattern having a line section on at least one part, using a lithographic method.

There is no particular limitation to the device, provided it is a device whose manufacture involves such a process, and examples include semiconductor devices, magnetic heads, micro lenses, liquid crystal elements, and the like.

To be specific, a reinforcing section may be provided on a line section of a photoresist pattern to be obtained. That is, a photoresist pattern 1 with reinforcing sections as described in the first embodiment is formed such that the line section 2 is a part, or the whole of, a line section of a photoresist pattern to be obtained.

At least one reinforcing section may be provided, and there is no particular limitation to the number of reinforcing sections and the spacing between the reinforcing sections, so that it is possible to arrange them as desired provided they contribute to preventing the line section collapsing and being washed away.

According to the present embodiment, even in a case of forming a photoresist pattern including a line section with a small line width, it is possible to prevent a part of the photoresist pattern from being washed away by the developing process, and the line shaped part from collapsing after the developing process. Thus it is possible to form an intended photoresist pattern accurately.

In addition, in the present embodiment, it is also possible to provide a sublayer as in the first embodiment if required.

### EXAMPLES

Hereunder are specific examples.

### Example 1

For a silicon substrate on which a sublayer soluble in a developing solution to be used in a later developing process was formed, a negative photoresist (trade name: TGMR-EN103PE, made by Tokyo Ohka Kogyo Co., Ltd.) was coated onto the sublayer, and prebaked under thermal conditions of 110° C. for 90 seconds to form a photoresist having a thickness of 0.2 μm.

Next, a photoresist film was drawn using an electron beam direct writing system (trade name: HL-800D, made by Hitachi Ltd.), and using a writing system programmed in advance such that pattern data could be created corresponding to a photoresist pattern 1 with reinforcing sections as shown in FIGS. 1 and 2. The line width D of the line section 2 in the photoresist pattern 1 with reinforcing sections was 100 nm, the length C of the line section 2 was 10 μm (10,000 nm), the maximum width A of the reinforcing section 3 was 5.0 μm, the length B of the reinforcing section 3 was 5.0 μm, the positional difference E1 (E2) was 5 μm (5000 nm), the spacing F between the photoresist pattern 1 with reinforcing sections was 10 μm, and the overall length of the photoresist pattern 1 with reinforcing sections was 10 mm. Furthermore, the number of photoresist pattern 1 with reinforcing sections formed on the substrate was three.

Subsequently, after performing a PEB process under thermal conditions of 120° C. for 90 seconds, a developing solution (TMAH, concentration 2.38 weight %) was dripped onto the substrate for the developing process. Then by rinsing, photoresist pattern 1 with reinforcing sections were formed. The period of the developing process was 30 seconds.

In this manner, three evaluation substrates were created under the same conditions.

The three evaluation substrates were each split by hand, and the cross sections were observed using a SEM. The result was that cross sections of the line sections 2 were contained in each of the cross sections of the three evaluation substrates. Furthermore, as a result of observing the cross sections, it was confirmed that there was no collapsing or being washed away of the line sections 2.

### Example 2

Evaluation substrates were created as described above in Example 1 except that the positional difference, E1 (E2), between reinforcing sections 3 was zero, with the locations of the reinforcing sections 3 being the same in the X direction.

Three evaluation substrates were created under the same conditions, and then each was split and the cross section observed using a SEM. As a result, two of the three evaluation substrates contained cross sections of line sections 2 in the cross section, but the other contained only a cross section of the reinforcing section 3 in the cross section. The result of observing the cross sections of the two whose cross sections of the line sections 2 could be observed, was that it was confirmed that there was no collapsing or being washed away of the line sections 2.

### Example 3

Evaluation substrates were created as described above in Example 1 except that the shape of the photoresist pattern 1 with reinforcing sections was changed to one without reinforcing sections 3. That is, three line shaped photoresist patterns with a line width of 100 nm and a length of 10 nm were formed on the sublayers.

Three evaluation substrates were created under the same conditions, and then each was split by hand and the cross sections observed using a SEM. The result was that no cross section of the photoresist patterns could be observed in the cross sections of any of the three evaluation substrates. This confirmed that the photoresist patterns were washed away in the developing process.

1. A photoresist pattern with a reinforcing section, wherein there is provided a line section and a reinforcing section that continues to said line section and that has a greater width than a line width of said line section.

2. A method of forming a photoresist pattern comprising; forming a photoresist pattern with a reinforcing section according to claim 1 by a process comprising forming on a substrate a photoresist film, exposing the photoresist film, and developing the photoresist film.

3. A method of forming a photoresist pattern according to claim 2, comprising forming on said substrate a sublayer film that is soluble in a developing solution used in said developing process, and forming said photoresist film on said sublayer film.

4. A method of evaluating a photoresist pattern, comprising forming on a substrate said photoresist pattern with a reinforcing section, using the method of forming a photoresist pattern according to either one of claim 2 and claim 3, to create an evaluation substrate, and splitting said evaluation substrate in a cross section perpendicular to the lengthwise direction of said line section to observe said cross section.

5. A method of evaluating a photoresist pattern according to claim 4, wherein when creating said evaluation substrate, a plurality of photoresist patterns with reinforcing sections is formed such that lengthwise directions of line sections are parallel, and locations of reinforcing sections in the lengthwise direction of the line sections are different for adjacent photoresist patterns with reinforcing sections.

6. A method of manufacturing a device using a lithographic method that includes a process of forming on a substrate a photoresist pattern having a line section on at least part thereof, wherein said photoresist pattern is formed such that there is provided on said line section a reinforcing section having a greater width than the line width of said line section, or a plurality of such reinforcing sections with spaces between them.

7. A mask used when exposing a photoresist film formed on a substrate, wherein a shape of either one of an exposure region and a non exposure region of said mask comprises a succession of line sections, and reinforcing sections having a greater width than a line width of said line sections.

8. A writing system used when drawing on a photoresist film formed on a substrate, which comprises an electron beam direct writing system that is provided with: a holding device for holding said substrate; an irradiating device for irradiating an electron beam onto said substrate; a moving device for moving a location on said substrate irradiated by said electron beam; and a control device for creating pattern data to determine an irradiation region of said electron beam on said substrate according to input information, and for controlling said moving device and said irradiating device based on said pattern data,
wherein the shape of the irradiation region of said electron beam is set to the shape of a succession of line sections, and reinforcing sections having a greater width than the line width of said line sections, and it is possible to input as said information, the line width of the line section, the length of the line section, the shape of the reinforcing section, the maximum width of the reinforcing section, and the length of the reinforcing section.
